(19) United States
(12) Patent Application Publication
(10) Pub. No.: US 2023/0201463 A1
(43) Pub. Date: Jun. 29, 2023

(54) Medicament Delivery System

(71) Applicants: Christopher J. Sawyer, Bennett, CO (US); James T. Sawyer, Bennett, CO (US); Joanna D. Meston, Bennett, CO (US); Stephen E. Muth, Bennett, CO (US)

(72) Inventors: Christopher J. Sawyer, Bennett, CO (US); James T. Sawyer, Bennett, CO (US); Joanna D. Meston, Bennett, CO (US); Stephen E. Muth, Bennett, CO (US)

(21) Appl. No.: 18/089,656

(22) Filed: Dec. 28, 2022

Related U.S. Application Data
(60) Provisional application No. 63/294,184, filed on Dec. 28, 2021.

(51) Int. Cl. A61M 5/20; A61M 5/315

(52) U.S. Cl. CPC A61M 5/204; A61M 5/31576; A61M 5/31593; A61M 2205/502; A61M 2205/3303

(57) ABSTRACT

A medicament delivery system is described. Embodiments of the medicament delivery system can include a smart user device and a medicament delivery assembly. The medicament delivery assembly can be implemented as a single-use cartridge that can be removably coupled to the smart user device. The smart user device can be configured to determine when to automatically administer a medicament stored in the medicament delivery assembly.

### CROSS-REFERENCE TO RELATED APPLICATION

This application claims the benefit of U.S. Provisional Application No. 63/294,184, filed Dec. 28^{th}, 2021.

### BACKGROUND

Currently available devices for providing a user quick administration of a medicament to counteract a medical issue include EpiPen®, AUVI-Q®, and a wearable device that detects apnea patterns and can administer a dose of naloxone based on apnea patterns.

Naloxone is a medicament that can prevent and/or reverse the effects of various opioids including respiratory depression and other indications that result from opioid toxicity if administered in a timely fashion. Since naloxone is typically introduced via injection, naloxone is generally delivered by a healthcare provider or a person trained to deliver the naloxone. However, access to a trained health care provider is not always possible when an individual is suffering from an overdose. When access to a trained professional is not available, a user or other party will need to introduce the naloxone to the affected user. In instances where the user is by themselves, there is a high risk that the naloxone will not be adequately delivered, if at all.

A low cost and reusable means for monitoring an individual that will inject a predetermined amount of naloxone to the individual based on determining the individual may need the naloxone is needed.

### BRIEF DESCRIPTION OF THE DRAWINGS

FIG. 1 is a block diagram of a medicament delivery system according to one embodiment of the present invention.

FIG. 2A is a perspective view of a smart user device according to one embodiment of the present invention.

FIG. 2B is a perspective view of a smart user device according to one embodiment of the present invention.

FIG. 2C is perspective view of a smart user device and a medicament delivery assembly according to one embodiment of the present invention.

FIG. 3 is a block diagram of a medicament delivery assembly according to one embodiment of the present invention.

FIG. 4A is a side view of a medicament delivery assembly according to one embodiment of the present invention.

FIG. 4B is a cross-sectional view of a medicament delivery assembly according to one embodiment of the present invention.

FIG. 4C is a perspective view of a medicament delivery assembly according to one embodiment of the present invention.

FIG. 5 is a perspective view of a medicament delivery assembly and an actuator according to one embodiment of the present invention.

FIG. 6A is a perspective view of a dual medicament delivery assembly according to one embodiment of the present invention.

FIG. 6B is a side view of a dual medicament delivery assembly according to one embodiment of the present invention.

FIG. 7 is flow diagram of a method for implementing a medicament delivery system according to one embodiment of the present invention.

FIG. 8 is flow diagram of a method for implementing a medicament delivery system including naloxone according to one embodiment of the present invention.

### DETAILED DESCRIPTION

Embodiments of the present invention include a medicament delivery system and method(s) of use. Typically, the medicament delivery system can include, but is not limited to, a smart user device and a medicament delivery assembly. The smart user device can include, but is not limited to, one or more sensors, a control module, and a power source. The medicament delivery assembly can be implemented as a cartridge removably received by the smart user device. Typically, each of the components of the smart user device can be contained in a housing that includes a receptacle for removably receiving the medicament delivery assembly therein. The housing can be attached to a strap that can be secured to an arm of a wearer. In some instances, the strap can be coupled to a wearer such that the wearer cannot remove the strap. For instance, a locking mechanism that can be only unlocked by a caregiver can be implemented. It is to be appreciated that the medicament delivery system can be referred to as the “ZEM Band”.

Data from the one or more sensors can be used to determine when to administer a medicament stored in the medicament delivery assembly. In a typical embodiment, the one or more sensors can include a first sensor and a second sensor. For example, the first sensor can be a pulse oximeter sensor and the second sensor can be an inertial sensor. The pulse oximeter sensor can be implemented to determine an oxygen saturation level of a wearer and the inertial sensor can be implemented to detect movement of the wearer.

In one embodiment, the medicament delivery assembly can include, but is not limited to, an autoinjector and a release mechanism. The autoinjector can include a medicament container, a piston, a spring, and a nozzle. The release mechanism can be operatively connected to the control module and the autoinjector. Typically, the autoinjector and the release mechanism can be housed in a single module and implemented as a cartridge.

The medicament container can be configured to store a medicament for injection into a wearer of the smart user device. For example, the medicament container can store a dose of naloxone. In another example, the medicament container may store one of insulin, epinephrine, atropine, diazepam, and vasopressin. As can be appreciated, an appropriate medicament can be selected based on the needs of a wearer. The autoinjector can be configured to inject the medicament stored in the medicament container into a wearer of the strap. In one example, the autoinjector can include a spring-loaded needle. In another example, the autoinjector can be gas powered. In yet another example, the autoinjector can be a jet injector. It is to be appreciated that one or more means of administering (or injecting) a medicament can be implemented without exceeding a scope of this disclosure. Of significant note, the medicament delivery system can be configured to receive a cartridge storing a medicament that can be injected. The medicament delivery system can be adapted to work with a variety of different medicaments with interchangeable cartridges.

Included hereinafter are a few protocols for when to administer and a how much of a particular medicament. It is to be appreciated that these protocols are a general guidance and not meant to be limiting.

In general, naloxone should be injected if there is a falling oxygen saturation with a good waveform (sine wave), low (or shallow) breathing to no breathing, a slow heartbeat, and/or low blood pressure. One or more of these indicators can be used a parameter by the control module to determine when to administer naloxone. In one example, an initial dose of 0.4 mg to 2.0 mg of naloxone may be administered. A falling oxygen saturation more the 10% below a baseline of a particular wearer, or an absolute value less than 80%, should be a parameter to immediately administer the naloxone. If there is no improvement detected, naloxone injections can be repeated in 2-3 minutes.

For cartridges containing epinephrine, a parameter for determining when to inject epinephrine can be severe bradycardia less than 35 and/or hypotension with systolic pressures below 80. The dose of epinephrine for anaphylaxis can be approximately 0.01 mg/kg (amount of epinephrine/weight of wearer) up to 0.30 mg/kg.

For cartridges containing atropine, a parameter for determining when to inject atropine can be heart rate less than 40 and falling. In another instance, a heart rate more than 10 beats less than normal low rate for the individual if normally around 40. For atropine treatment of organophosphorus poisoning (also for bradycardia from any cause): Adults and children older than 10 years of age weighing more than 41 kilograms-2 milligrams injected into a muscle; Children 4 to 10 years of age weighing 18 to 41 kg-1 mg injected into a muscle.

Vasopressin is a drug that can be used in pulseless arrest or for hypotension. For diabetes insipidus, 10 units can be delivered subcutaneous or intramuscular. The dose for pulseless cardiac arrest is 40 units intravenous.

Embodiments are contemplated where the cartridge can include diazepam and the smart user device can include at least an ultrasonic sensor. As known, diazepam can be used to treat seizures. A dose of 5-10 mg of diazepam may be included in a cartridge. Seizure symptoms may be detected by the ultrasonic sensor.

Embodiments are contemplated where sensors specific to a potentially fatal diagnosis can be implemented. The control module can be adapted to analyze data from the one or more sensors to determine when administration of a particular medicament may be needed. For instance, a sensor designed to analyze sweat secreted from a wearer is contemplated. The control module would then be able to analyze the sweat to detect drug and/or alcohol use. The data from the sensor could be analyzed by the control module to determine if a medicament should be administered.

In a typical implementation, the release mechanism can be activated by the control module. In some embodiments, the medicament delivery system can include a means for manually activating the autoinjector by a wearer of the smart user device. For instance, the medicament delivery assembly can include a mechanical actuator to engage the release mechanism that can be actuated by the user. In one example, the smart user device can include a linear actuator that can be activated by the control module. The linear actuator can be adapted to engage the release mechanism to active the autoinjector. In some instances, the smart user device can be connected to a wireless network. The smart user device can be configured to receive a signal (or command) via the wireless network to activate the autoinjector. For example, a caregiver may determine that the medicament needs to be injected and can remotely activate the autoinjector via the wireless network.

In one example embodiment where naloxone is provided as the medicament, the control module can be configured to receive signals from the pulse oximeter sensor and the inertial sensor to determine when to activate the autoinjector of the medicament delivery assembly. For instance, the control module can send a signal to the release mechanism to activate the autoinjector based on determining that an oxygen level is below a predetermined threshold and the inertial sensor has not detected any movement for a predetermined amount of time. Of significant note, by determining an optimum time to inject the medicament by monitoring the two sensors, the wearer of the strap can have the best chance for overcoming an overdose. The control module can be programmed to activate the autoinjector based on data received from one or more of the sensors. For instance, the control module can be configured to activate the autoinjector based on receiving data from the pulse oximeter that indicates an oxygen saturation level has dropped below a critical level. In this instance, the control module may activate the autoinjector even when receiving data from the inertial sensor that there is movement. In another instance, the control module can be configured to send a signal to the release mechanism based on determining there is no movement detected by the inertial sensor for a predetermined amount of time.

In one embodiment, the medicament delivery assembly may be located on a different part of the body than the strap. For instance, where insulin is in the medicament container for a Diabetic, the medicament delivery assembly can be secured to a thigh of a user. The control module can be configured to send a wireless signal to the medicament delivery assembly to activate the autoinjector.

Embodiments are contemplated where the medicament delivery assembly includes more than one medicament. In such an embodiment, the medicament delivery assembly can include, but is not limited to, a first autoinjector, a first release mechanism, a second autoinjector, and a second release mechanism. The first release mechanism can be operatively connected to the first autoinjector and the second release mechanism can be operatively connected to the second autoinjector. The control module can be configured to determine a medicament stored in each of the autoinjectors along with determining one or more protocols to follow based on the medicaments. For instance, the autoinjectors may each contain insulin. A wearer would then have two doses of insulin ready for injection when needed. The control module can be configured to determine that both autoinjectors include insulin in the medicament delivery assembly. The control module may then start a predetermined protocol for a medicament delivery assembly containing two doses of insulin. Instances are contemplated where the dual medicament delivery assembly may include different medicaments. In such an instance, the control module can be configured to run two different protocols simultaneously to determine when to administer either of the medicaments based on data from the sensors.

In some instances, a remotely located database can be implemented to store data from the smart user device. The smart user device can be configured to send data to the remotely located database via a wireless network. For instance, physiological data measured by the one or more sensors, and analyzed by the control module, can be sent to the remote database. Authorized users can access the remote database to monitor physiological data of a wearer. For example, a recently released patient from alcohol and/or drug rehabilitation may be required to wear a medicament delivery system. A caregiver(s) can access data from the remote database via an application or web interface to monitor the patient. Embodiments are contemplated where a caregiver may monitor the data in near real time. The caregiver may have the authority to activate the autoinjector (via the control module) remotely based on monitoring the data from the smart user device. For example, if a wearer is hovering near the thresholds for the autoinjector to be engaged, but not quite getting to the threshold, a caregiver can send a signal to the smart user device to activate the release mechanism to inject the wearer. In another example, a caregiver can be given permission to have the medicament delivery system administer a medicament based on their judgement. Embodiments are contemplated where a caregiver, having access to the remote database, can alter parameters for administrating a medicament for a particular wearer. For example, a caregiver can adjust parameters for injection based on one or more physical characteristics of a wearer.

In one embodiment, a medicament delivery system can include, but is not limited to, a cartridge and a smart user device. The cartridge can include an autoinjector and a release mechanism. The autoinjector can contain a medicament. The release mechanism can be operatively connected to the autoinjector. The smart user device can be adapted to operatively connect to the cartridge and can include a housing, a control module, an inertial sensor, and a pulse oximeter sensor. The housing can (i) be coupled to a strap, and (ii) include a receptacle for removably receiving the cartridge therein. The control module can be (i) operatively connected to the cartridge when the cartridge is inserted into the receptacle, and (ii) adapted to determine the medicament when the cartridge is inserted into the receptacle. The inertial sensor and the pulse oximeter sensor can each be adapted to send data to the control module.

In another embodiment, a medicament delivery system can include, but is not limited to, a cartridge and a smart user device. The cartridge can include a primary autoinjector containing a first medicament, a primary release mechanism operatively connected to the primary autoinjector, a secondary autoinjector containing a second medicament, and a secondary release mechanism operatively connected to the secondary autoinjector. The smart user device can be adapted to operatively receive the cartridge and can include a housing, a control module, an inertial sensor, and a pulse oximeter sensor. The housing can (i) be coupled to a strap, and (ii) include a receptacle for removably receiving the cartridge therein. The control module can be (i) operatively connected to the cartridge when the cartridge is inserted into the receptacle, and (ii) adapted to determine the first medicament and the second medicament when the cartridge is inserted into the receptacle. The inertial sensor and the pulse oximeter sensor can each be adapted to send data to the control module.

In yet another embodiment, a medicament delivery system can include, but is not limited to, a cartridge and a smart user device. The cartridge can include a primary autoinjector containing a first medicament, a primary release mechanism operatively connected to the primary autoinjector, a secondary autoinjector containing a second medicament, and a secondary release mechanism operatively connected to the secondary autoinjector. The smart user device can be adapted to operatively receive the cartridge and can include a housing, a control module, a linear actuator, a first sensor, and a second sensor. The housing can (i) be coupled to a strap, and (ii) include a receptacle for removably receiving the cartridge therein. The linear actuator can be adapted to engage the primary release mechanism and the secondary release mechanism. The control module can be (i) operatively connected to the linear actuator, and (ii) adapted to determine the first medicament and the second medicament when the cartridge is inserted into the receptacle. The first sensor and the second sensor can each be adapted to send data to the control module.

Embodiments are contemplated where the medicament delivery system can be implemented to routinely provide medicament injections based on a predetermined schedule. For instance, where a wearer needs to routinely take a medicament for one or more medical conditions, the medicament delivery system can be implemented to provide the wearer with reminders and administration of the medicament as needed. Generally, the control module can implement a protocol based on a regimen for the medicine to be administered. The smart user device can be adapted to present an alert to a wearer letting them know that a dose of medicament is needed and then administer the medicament to the wearer. Of note, the smart user device can alert a wearer that a new cartridge is needed after the medicament has been administered. Further, the smart user device can alert a wearer if a new cartridge is needed after determining that a loaded cartridge does not have any medicament.

The present invention can be embodied as devices, systems, methods, and/or computer program products. Accordingly, the present invention can be embodied in hardware and/or in software (including firmware, resident software, micro-code, etc.). Furthermore, the present invention can take the form of a computer program product on a computer-usable or computer-readable storage medium having computer-usable or computer-readable program code embodied in the medium for use by or in connection with an instruction execution system. In one embodiment, the present invention can be embodied as non-transitory computer-readable media. In the context of this document, a computer-usable or computer-readable medium can include, but is not limited to, any medium that can contain, store, communicate, propagate, or transport the program for use by or in connection with the instruction execution system, apparatus, or device.

The computer-usable or computer-readable medium can be, but is not limited to, an electronic, magnetic, optical, electromagnetic, infrared, or semiconductor system, apparatus, device, or propagation medium.

### Terminology

The terms and phrases as indicated in quotation marks (“ ”) in this section are intended to have the meaning ascribed to them in this Terminology section applied to them throughout this document, including in the claims, unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive; rather the term is inclusive, meaning either or both.

References in the specification to “one embodiment”, “an embodiment”, “another embodiment, “a preferred embodiment”, “an alternative embodiment”, “one variation”, “a variation” and similar phrases mean that a particular feature, structure, or characteristic described in connection with the embodiment or variation, is included in at least an embodiment or variation of the invention. The phrase “in one embodiment”, “in one variation” or similar phrases, as used in various places in the specification, are not necessarily meant to refer to the same embodiment or the same variation.

The term “couple” or “coupled” as used in this specification and appended claims refers to an indirect or direct physical connection between the identified elements, components, or objects. Often the manner of the coupling will be related specifically to the manner in which the two coupled elements interact.

The term “directly coupled” or “coupled directly,” as used in this specification and appended claims, refers to a physical connection between identified elements, components, or objects, in which no other element, component, or object resides between those identified as being directly coupled.

The term “approximately,” as used in this specification and appended claims, refers to plus or minus 10% of the value given.

The term “about,” as used in this specification and appended claims, refers to plus or minus 20% of the value given.

The terms “generally” and “substantially,” as used in this specification and appended claims, mean mostly, or for the most part.

Directional and/or relationary terms such as, but not limited to, left, right, nadir, apex, top, bottom, vertical, horizontal, back, front and lateral are relative to each other and are dependent on the specific orientation of a applicable element or article, and are used accordingly to aid in the description of the various embodiments and are not necessarily intended to be construed as limiting.

The term “software,” as used in this specification and the appended claims, refers to programs, procedures, rules, instructions, and any associated documentation pertaining to the operation of a system.

The term “firmware,” as used in this specification and the appended claims, refers to computer programs, procedures, rules, instructions, and any associated documentation contained permanently in a hardware device and can also be flashware.

The term “hardware,” as used in this specification and the appended claims, refers to the physical, electrical, and mechanical parts of a system.

The terms “computer-usable medium” or “computer-readable medium,” as used in this specification and the appended claims, refers to any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium. By way of example, and not limitation, computer readable media may comprise computer storage media and communication media.

The term “signal,” as used in this specification and the appended claims, refers to a signal that has one or more of its characteristics set or changed in such a manner as to encode information in the signal. It is to be appreciated that wireless means of sending signals can be implemented including, but not limited to, Bluetooth, Wi-Fi, acoustic, RF, infrared and other wireless means.

### An Embodiment of a Medicament Delivery System

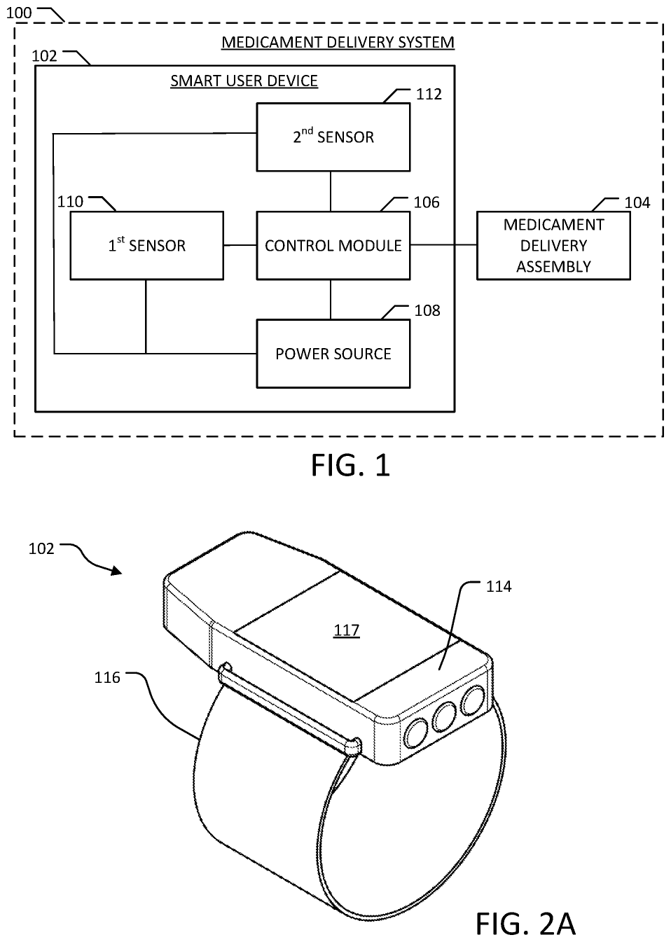

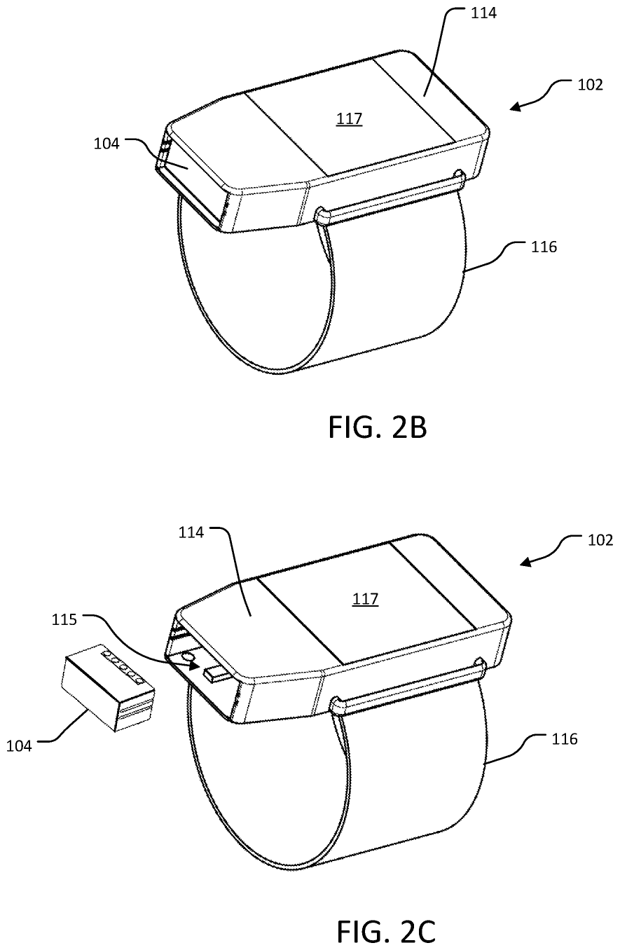

Referring to FIG. 1, a block diagram of a first embodiment 100 of a medicament delivery system is illustrated. The medicament delivery system 100 can be implemented to auto-inject a medicament without user action based on one or more physiological measurements being monitored by the medicament delivery system 100. Cartridges having different medicaments can be implemented with the medicament delivery system 100. FIGS. 2A-2C include detailed diagrams of one embodiment of a smart user device and medicament delivery assembly of the medicament delivery system 100. FIG. 2A-B includes a view of the smart user device with a medicament delivery assembly and FIG. 2C includes a view of the smart user device without the medicament delivery assembly.

As shown in FIG. 1, the medicament delivery system 100 can include, but is not limited to, a smart user device 102 and a medicament delivery assembly 104. The smart user device 102 can include, but is not limited to, a control module 106, a power source 108, a first sensor 110, and a second sensor 112.

The smart user device 102 can have a hardware platform and software components. In general, the hardware platform can include a processor, a user interface, storage, and a display. The processor can be a single microprocessor, multi-core processor, or a group of processors. The user interface can include one or more buttons and the display when implemented as a touch display. The storage can include random access memory and nonvolatile storage. The random access memory can store executable code as well as data that can be immediately accessible to the processor. The nonvolatile storage can store executable code and data in a persistent state. Typically, the display can be a touch display configured to receive input from a user. The software components of the smart user device 102 can include one or more databases which can store information related to various medicaments that the smart user device 102 may be used to administer. The one or more databases can also store methodologies (or protocols) for determining when to administer a particular medicament based on data received from the sensors 110/112. The software components can also include an operating system on which various applications can execute.

As shown generally in FIGS. 2A-2C, the smart user device 102 can typically include a housing 114 to store each of the components, a strap 116, and a display 117. The strap 116 can be implemented to secure the smart user device 102 to a wearer. In one example, the strap 116 can be implemented to secure the medicament delivery system 100 to a wrist of a wearer. The strap 116 can generally be comprised of a durable material that can be worn on a wrist without causing pain to the wearer. For instance, synthetic rubber can be implemented to manufacture the strap 116. As previously mentioned, the display 117 can typically be a touch display configured to present graphics to a wearer and a means for input by the user.

As shown in FIG. 2C, the housing 114 of the smart user device 102 may include a receptacle 115 for receiving the medicament delivery assembly 104 therein. For instance, the medicament delivery assembly 104 can be operatively loaded into the receptacle 115 of the housing 114. The receptacle 115 can include a means for operatively connecting the medicament delivery assembly 104 to the control module 106. Of note, the medicament delivery assembly 104 can include a means for identifying a medicament in the medicament delivery assembly 104 such that the control module 106 can determine the medicament when the medicament delivery assembly 104 may be inserted into the receptacle 115. In some instances, the smart user device 102 may request that a user confirm the medicament being provided in the medicament delivery assembly 104.

The control module 106 can include a means for determining a type of medicament in a particular cartridge 104. For instance, a first cartridge containing naloxone can be inserted into the smart user device 102. The control module 106 can determine a naloxone cartridge has been inserted and can automatically update how the control module 106 will determine when to inject the naloxone. For instance, a predetermined protocol can be implemented once the control module 106 determines the inserted cartridge includes naloxone. In another instance, a second cartridge can be inserted that contains Insulin. The control module 106 can determine the second cartridge contains Insulin and implement a new protocol predetermined for an Insulin containing cartridge.

Referring back to FIG. 1, the medicament delivery assembly 104, the first sensor 110, and the second sensor 112 can each be operatively connected to the control module 106. The power source 108 can be implemented to provide power to the components of the medicament delivery system 100.

Typically, the power source 108 can be a rechargeable power source. Embodiments are contemplated where the power source 108 is removable and replaceable with a second power source. For instance, a small rechargeable battery bank can be implemented such that the strap 116 can be worn while replacing the power source 108.

The first sensor 110 and the second sensor 112 can be implemented to measure one or more physiological conditions and provide data to the control module 106. Typically, the sensors 110/112 can be adapted for a specific purpose and send appropriate signals to the control module 106 to analyze. In one instance, the first sensor 110 can be a pulse oximeter sensor configured to measure an oxygen saturation level in a wearer and the second sensor 112 can be an inertial sensor configured to determine if a wearer is moving. For example, the control module 106 can receive data from the first sensor 110 and determine a current oxygen saturation level for the wearer. In some instances, the control module 106 may continuously analyze the data from the first sensor 110. In other instances, the control module 106 may analyze data in predetermined intervals. The control module 106 can be configured to determine when to administer the medicament in the medicament delivery assembly 104 based on data received from the first sensor 110 and the second sensor 112. In some instances, the control module 106 may determine to administer the medicament based solely on one of the sensors 110/112.

The pulse oximeter sensor 110 can be implemented to help determine an oxygen saturation level of a wearer. In one example, the pulse oximeter sensor 110 can implement reflectance pulse oximetry to measure oxygen saturation levels. It is to be appreciated that other means of measuring oxygen saturation can be implemented without exceeding a scope of the present invention. The pulse oximeter sensor 110 can be operatively connected to the control module 106 to send data to the control module 106. The control module 106 can be configured to receive the data and determine an oxygen saturation level based on the data received. The control module 106 can further determine if the analyzed oxygen saturation level is below or above a predetermined threshold. In some instances, the control module 106 can average several calculations of the oxygen saturation level based on data received from the pulse oximeter sensor 110 at different times. For example, the control module 106 may average two or more data sets from data received 30 seconds apart.

The inertial sensor 112 can typically include at least one accelerometer and at least one gyroscope. Embodiments of the inertial sensor 112 can include other sensors adapted to measure acceleration and directional orientation. The inertial sensor 112 can be operatively connected to the control module 106 to send data to the control module 106 of which the control module 106 can determine movement. In some instances, the inertial sensor 112 can send data in set intervals to help conserve battery life of the smart user device 102.

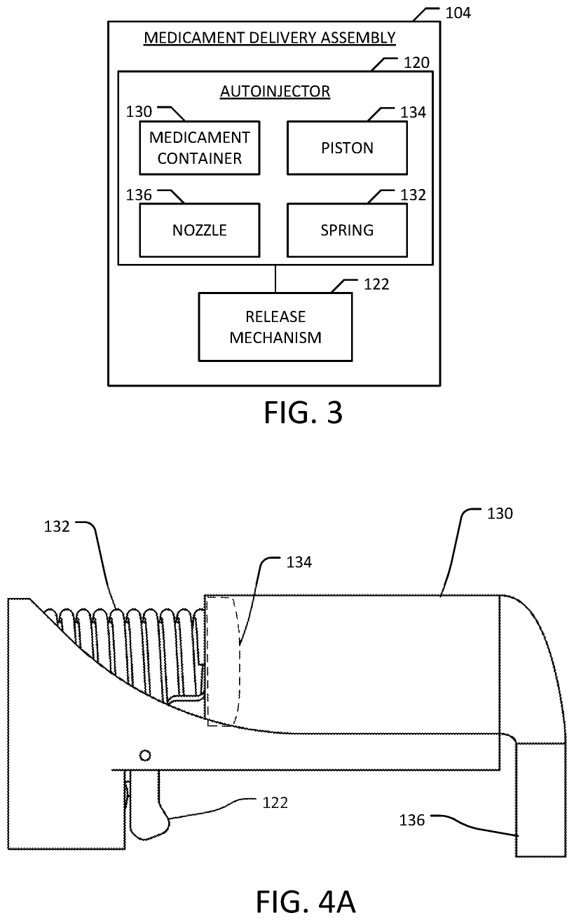

Referring to FIG. 3, a block diagram of one embodiment of the medicament delivery assembly 104 is illustrated. The medicament delivery assembly 104 can be assembled as a cartridge that may be removably coupled to the smart user device 102. As previously mentioned, the smart user device 102 can include a receptacle 115 for receiving the cartridge 104 therein.

As shown, the cartridge 104 can include, but is not limited to, an autoinjector 120 and a release mechanism 122. The release mechanism 122 can be operatively connected to the autoinjector 120. Generally, the control module 106 can send a signal to an actuator to engage the release mechanism 122 and activate the autoinjector 120. In one instance, the autoinjector 120 can be a jet injector. When the release mechanism 122 may be activated by the control module 106, the release mechanism 122 can mechanically activate the autoinjector 120 which can inject contents of the autoinjector 120 into the wearer of the medicament delivery system 100.

In one embodiment, the autoinjector 120 can include, but is not limited to, a medicament container 130, a spring 132, a piston 134, and a nozzle 136. The medicament container 130 can be configured to store a medicament and can include a first opening and a second opening. The spring 132 can typically be a compression spring that can be located proximate the piston 134. The compression spring 132 can be operatively connected to the release mechanism 122 such that the release mechanism 122 can be implemented to release the compression spring 132 under compression. The piston 134 can be located inside the first opening of the medicament container 130. A first side of the piston 134 can engage the compression spring 132 and a second side can engage the medicament in the medicament container 130. Of note, the piston 134 can be sized to frictionally fit within the medicament container 130 such that the medicament does not leak out of the piston side of the medicament container 130. The nozzle 136 can be fluidly connected to the second opening of the medicament container 130. In one instance, the nozzle 136 may include a seal that can be implemented to prevent the medicament from leaking. When the compression spring 132 may be released pushing the piston 134 through the medicament container 130, the seal may be broken allowing the medicament to exit the nozzle 136. As can be appreciated, a size of an opening of the nozzle 136 can be adjusted based on a medicament being administered. For instance, a viscosity of the medicament may be used to determine a nozzle opening size.

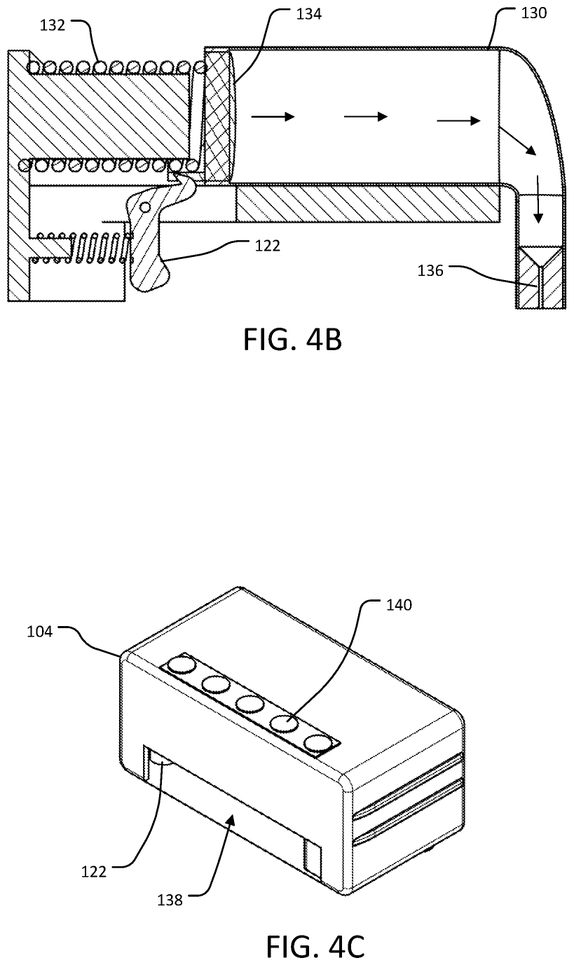

Referring to FIG. 4A, a side view of one example embodiment of the cartridge 104 with a housing removed is illustrated. As previously mentioned, the cartridge 104 can be removably coupled to the smart user device 102. When the cartridge 104 is inserted into the receptacle 115 of the smart user device 102, the cartridge 104 can be operatively connected to the control module 106.

Of note, a jet injector can generally be shown in FIG. 4A. It is to be appreciated that other means of administering the medicament container 130 from the autoinjector 120 are contemplated. For instance, sharps can be implemented. As shown, the autoinjector 120 can include the medicament container 130, the compression spring 132, the piston 134, and the nozzle 136. The spring 132 can be implemented to drive the piston 134 forwards, forcing medicament in the medicament container 130 to be pushed through the nozzle 136.

Referring to FIG. 4B, a cross-sectional view of the cartridge 104 is illustrated. Of note, a flow path of the medicament and the piston 134 are shown. The piston 134 can pass through the medicament container 130 pushing a medicament out of the nozzle 136.

Referring to FIG. 4C, a back perspective view of the cartridge 104 is illustrated. Of note, the cartridge 104 can include an opening 138 wherein a component from the smart user device 102 may interface with the release mechanism 122. In one embodiment, the cartridge 104 can include an electrical connector mechanism 140. The electrical connector mechanism 140 can interface with the control module 106 allowing the control module 106 to determine a medicament in the cartridge 104. Typically, the electrical connector mechanism 140 can be configured (or coded) for a medicament in a particular cartridge. For instance, each cartridge including naloxone can have the same electrical connector mechanism configuration. In one instance, metal pins having a particular pattern can be implemented. For example, POGO pins can be implemented to code the cartridge 104 allowing the control module 106 to determine the medicament in the cartridge 104.

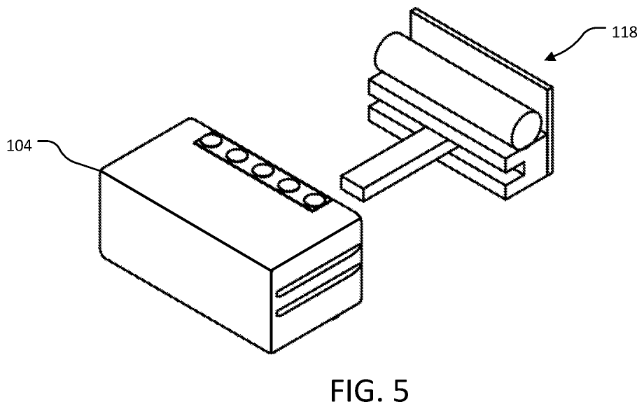

Referring to FIG. 5, a perspective view of the cartridge 104 and an engagement mechanism 118 is illustrated. In one example, the engagement mechanism 118 can be a linear actuator. The control module 106 can be operatively connected to the linear actuator 118 to engage the release mechanism 122 of the cartridge 104. When the cartridge 104 is inserted into the receptacle 115, the linear actuator 118 can pass through the opening 138 in the cartridge 104. In a typical implementation, the control module 106 can activate the linear actuator 118 to engage the release mechanism 122. The release mechanism 122 can be configured to release the spring 132 when engaged by the linear actuator 118. The stored kinetic energy in the compression spring 132 can be forceful enough to push the piston 134 substantially through a length of the medicament container 130. The piston 134 can push the medicament towards the nozzle 136. The nozzle 136 can be implemented to accelerate and disperse the medicament into a dermis layer of a wearer of the medicament delivery system 100. The medicament stored in an autoinjector can be selected for a particular use. Of note, the cartridge 104 can be implemented as a single-use cartridge which may be disposed of after the medicament has been administered.

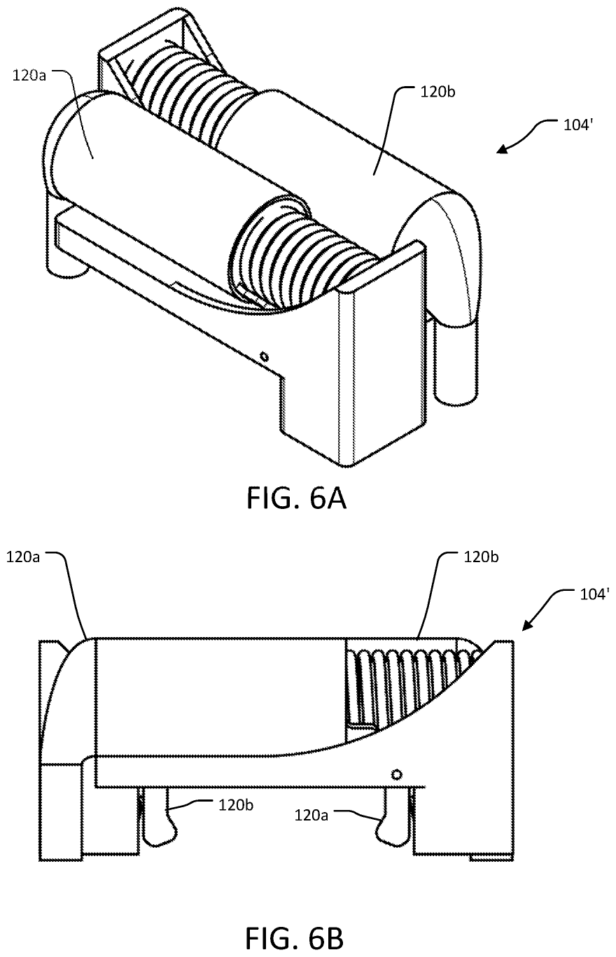

Referring to FIG. 6A-6B, different views of a dual medicament cartridge 104′ without a housing are illustrated. FIG. 6A includes a perspective view and FIG. 6B includes a side view of the dual medicament cartridge 104′. Of significant note, the dual medicament cartridge 104′ can include a first (or primary) autoinjector 120a, a first (or primary) release mechanism 122a, a second (or secondary) autoinjector 120b, and a second (secondary) release mechanism 122b. As can be appreciated, the dual medicament cartridge 104′ can be implemented to administer two medicaments to a wearer. The smart user device 102 can be operatively connected to the first release mechanism 122a and the second release mechanism 122b when the dual medicament cartridge 104′ may be inserted into the receptacle 115 of the smart user device 102.

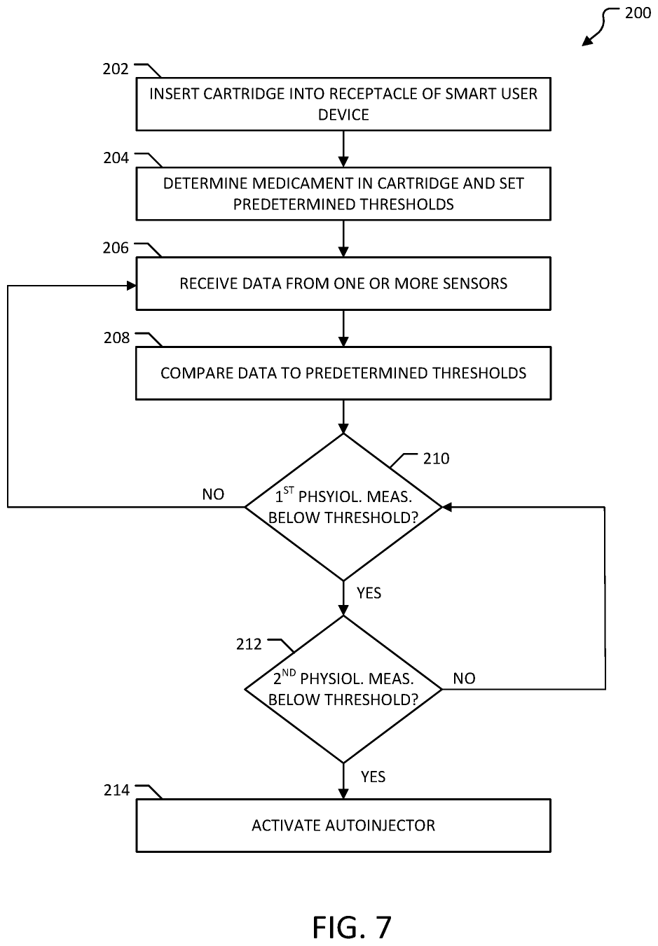

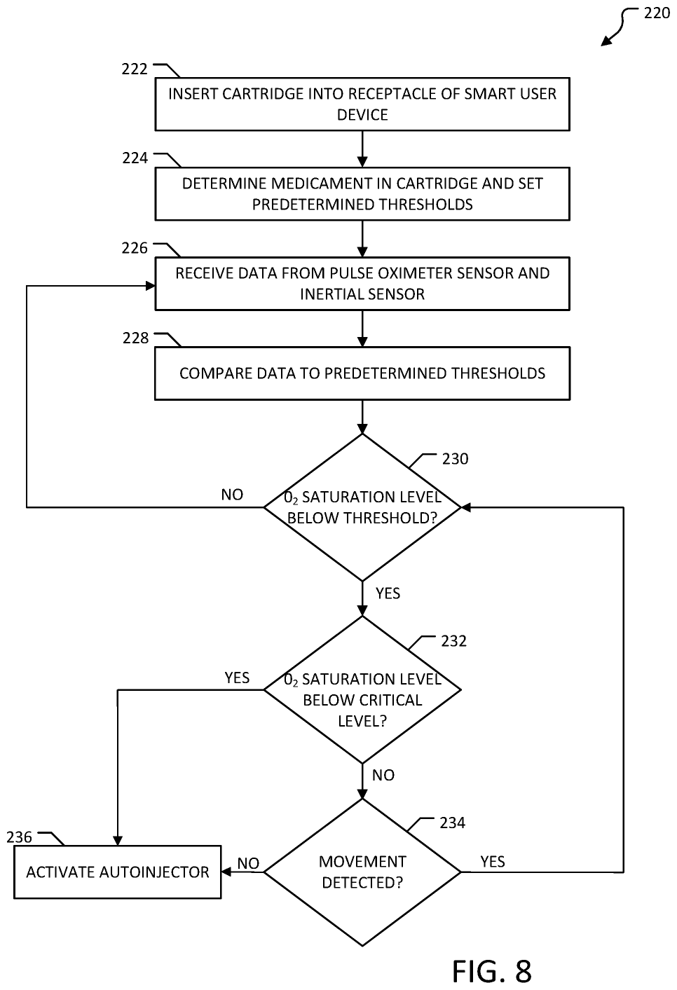

Referring to FIG. 7, a flow diagram of a method (or process) 200 for implementing the medicament delivery system 100 to determine when to provide a medicament to a wearer is shown. Generally, a wearer can secure the strap 116 to their wrist and a cartridge can be inserted into the receptacle 115 to operatively connect the cartridge to the smart user device 102. It is to be appreciated that the cartridge may have been previously inserted into the receptacle 115 of the smart user device 102.

After the strap 116 has been secured to a wearer, the wearer can turn the smart user device 102 “ON” which can then power on the display, the control module 106, the first sensor 110, and the second sensor 112. The first sensor 110 and the second sensor 112 can then start sending data to the control module 106.

In block 202, a wearer can insert a cartridge into the receptacle 115 of the smart user device 102. It is to be appreciated that a cartridge may already be inserted into the receptacle 115 and operatively connected to the smart user device 102.

In block 204, the control module 106 can determine the type of medicament stored in the cartridge and can load an appropriate application (or program) based on the type of medicament and sensors available to the control module 106. The selected application can contain a protocol having predetermined thresholds for the control module 106 to determine whether or not to administer the medicament based on data from the sensors 110/112. Of note, the control module 106 can be configured to load an appropriate application based on the medicament to be administered to a user. In some instances, the wearer may confirm the medicament in the cartridge after the cartridge has been inserted into the receptacle 115.

In block 206, the control module 106 can receive data from one or more sensors. The control module 106 can be configured to calculate appropriate physiological measurements from data received from the one or more sensors.

In block 208, the control module 106 can compare the data received to predetermined thresholds for physiological data measurable by the one or more sensors. Typically, the control module 106 can include predetermined thresholds for a minimum (or maximum) value for calculated physiological measurements. In one example, a pulse oximeter sensor can be implemented to measure oxygen saturation levels. The control module 106 may then determine if a calculated oxygen saturation level as determined from data from the pulse oximeter sensor is below a predetermined threshold.

In decision block 210, the control module 106 can determine if a first physiological measurement is below (or above) the predetermined threshold. If not, the process can move back to block 206. If the first physiological measurement is below (or above) the predetermined threshold, the process can move to decision block 212.

In decision block 212, the control module 106 can determine if a second physiological measurement is below (or above) the predetermined threshold. If not, the process can move back to block 206 to check the first physiological measurement again. If the second physiological measurement is below (or above) the predetermined threshold, the process can move to block 214.

In block 214, the control module 106 can activate the release mechanism 122 to activate the autoinjector 120. The autoinjector 120 can then administer the medicament to the wearer.

Embodiments are contemplated where the medicament delivery system 100 can alert authorities (e.g., 911 or a physician) that the medicament has been administered to the wearer.

Referring to FIG. 7, a flow diagram of a method (or process) 220 for implementing the medicament delivery system 100 to determine when to provide a naloxone to a wearer is shown. Generally, a wearer can secure the strap 116 to their wrist and a cartridge having naloxone can be inserted into the receptacle 115 to operatively connect the cartridge to the smart user device 102. It is to be appreciated that the cartridge may have been previously inserted into the receptacle 115 of the smart user device 102.

After the strap 116 has been secured to a wearer, the wearer can turn the smart user device 102 “ON” which can then power on the display, the control module 106, the first sensor 110, and the second sensor 112. The first sensor 110 can be a pulse oximeter sensor and the second sensor 112 can be an inertial sensor.

In block 222, a wearer can insert a cartridge having naloxone into the receptacle 115 of the smart user device 102. It is to be appreciated that a cartridge may already be inserted into the receptacle 115 and operatively connected to the smart user device 102.

In block 224, the control module 106 can determine the type of medicament stored in the cartridge and can load an appropriate application (or program) based on the type of medicament and sensors available to the control module 106. The selected application can contain a protocol having predetermined thresholds for the control module 106 to determine whether or not to administer the medicament based on data from the sensors 110/112. Based on the cartridge containing naloxone, the control module 106 can be configured to load an application allowing the control module 106 to determine when to administer the naloxone based on data from the sensors 110/112.

In block 226, the control module 106 can receive data from the pulse oximeter sensor 110 and the inertial sensor 112. The control module 106 can calculate an oxygen saturation level based on data from the pulse oximeter sensor 110 and can determine if the wearer is moving based on data received from the inertial sensor 112.

In block 228, the control module 106 can compare the data received to predetermined thresholds for oxygen saturation level and movement. Typically, the protocol, implemented based on naloxone being in the cartridge, implemented by the control module 106 can include predetermined thresholds for a minimum oxygen saturation level and a minimum threshold for movement within a certain timeframe.

In decision block 230, the control module 106 can determine if the calculated oxygen saturation level is below the predetermined threshold. If not, the process can move back to block 226. If the calculated oxygen saturation level is below the predetermined threshold, the process can move to decision block 232. In one example, the predetermined threshold can be based on a breathing rate of a wearer. Of note, a breathing rate can be approximated from data received from the pulse oximeter sensor. In such an embodiment, a breathing rate less than 12 breaths per minute can be the threshold. In embodiments where oxygen saturation levels are calculated, 90% oxygen saturation level can be the threshold.

In decision block 232, the control module 106 can determine if the calculated oxygen saturation level is below a critical level. For example, an oxygen saturation level below 85% can be the critical level. If the calculated oxygen saturation level is below the critical level, the process 220 can move to block 236. If the calculated oxygen saturation level is above the critical level, the process 220 can move to block 236. It is to be appreciated that the provided levels are approximate and not exact.

In decision block 234, the control module 106 can determine if the inertial sensor 112 has detected any movement from the wearer within a predetermined timeframe. If movement has been detected within the timeframe, the process 200 can move back to block 206 to check the oxygen saturation levels again. If no movement has been detected, the process can move to block 236.

In block 236, the control module 106 can activate the release mechanism 122 to activate the autoinjector 120. The autoinjector 120 can then administer the wearer with naloxone.

Embodiments are contemplated where the medicament delivery system can alert authorities (e.g., 911 or a physician) that the medicament has been administered to the wearer.

### ALTERNATIVE EMBODIMENTS AND VARIATIONS

The various embodiments and variations thereof, illustrated in the accompanying Figures and/or described above, are merely exemplary and are not meant to limit the scope of the invention. It is to be appreciated that numerous other variations of the invention have been contemplated, as would be obvious to one of ordinary skill in the art, given the benefit of this disclosure. All variations of the invention that read upon appended claims are intended and contemplated to be within the scope of the invention.

I claim:

1. A medicament delivery system comprising:
a cartridge including:
an autoinjector containing a medicament; and
a release mechanism operatively connected to the autoinjector;
a smart user device adapted to operatively connect to the cartridge, the smart user device including:
a housing (i) coupled to a strap, and (ii) including a receptacle for removably receiving the cartridge therein;
a control module (i) operatively connected to the cartridge when the cartridge is inserted into the receptacle, and (ii) adapted to determine the medicament when the cartridge is inserted into the receptacle;
an inertial sensor adapted to send data to the control module; and
a pulse oximeter sensor adapted to send data to the control module.

2. The medicament delivery system of claim 1, wherein the smart user device further includes a display.

3. The medicament delivery system of claim 1, wherein the control module determines when to activate the autoinjector based on data received from the inertial sensor and the pulse oximeter sensor.

4. The medicament delivery system of claim 1, wherein the smart user device further includes an actuator adapted to be (i) operatively connected to the release mechanism, and (ii) activated by the control module.

5. The medicament delivery system of claim 1, wherein the autoinjector includes:
a medicament container storing the medicament, the medicament container having
a first opening and a second opening;
a compression spring located proximate the first opening of the medicament container, the compression spring operatively connected to the release mechanism;
a piston having a first end engaging the compression spring and a second end engaging the medicament inside the medicament container; and
a nozzle coupled to the second opening of the medicament container.

6. The medicament delivery system of claim 5, wherein the cartridge is removed from the receptacle after the medicament is dispensed.

7. The medicament delivery system of claim 6, wherein a second cartridge having a second medicament is inserted into the receptacle after the cartridge is removed.

8. The medicament delivery system of claim 7, wherein the control module determines the second medicament in the second cartridge after the second cartridge is inserted into the receptacle.

9. The medicament delivery system of claim 8, wherein the control module is adapted to administer the second medicament based on a predetermined protocol based on the second medicament.

10. The medicament delivery system of claim 9, wherein the predetermined protocol is based on (i) the second medicament and (ii) data received from the pulse oximeter sensor and the inertial sensor.

11. A medicament delivery system comprising:
a cartridge including:
a primary autoinjector containing a first medicament;
a primary release mechanism operatively connected to the primary autoinjector;
a secondary autoinjector containing a second medicament; and
a secondary release mechanism operatively connected to the secondary autoinjector;
a smart user device adapted to operatively receive the cartridge, the smart user device including:
a housing (i) coupled to a strap, and (ii) including a receptacle for removably receiving the cartridge therein;
a control module (i) operatively connected to the cartridge when the cartridge is inserted into the receptacle, and (ii) adapted to determine the first medicament and the second medicament when the cartridge is inserted into the receptacle;
an inertial sensor adapted to send data to the control module; and
a pulse oximeter sensor adapted to send data to the control module.

12. The medicament delivery system of claim 11, wherein the primary autoinjector includes:
a primary medicament container storing the first medicament, the primary medicament container having a first opening and a second opening;
a primary compression spring located proximate the first opening of the primary medicament container, the primary compression spring operatively connected to the primary release mechanism;
a primary piston having a first end engaging the primary compression spring and a second end engaging the first medicament inside the primary medicament container; and
a primary nozzle coupled to the second opening of the primary medicament container.

13. The medicament delivery system of claim 11, wherein the secondary autoinjector includes:
a secondary medicament container storing the second medicament, the secondary medicament container having a first opening and a second opening;
a secondary compression spring located proximate the first opening of the secondary medicament container, the secondary compression spring operatively connected to the secondary release mechanism;
a secondary piston having a first end engaging the secondary compression spring and a second end engaging the second medicament inside the secondary medicament container; and
a secondary nozzle coupled to the second opening of the secondary medicament container.

14. The medicament delivery system of claim 11, wherein the control module is adapted to administer the first medicament based on a predetermined first protocol based on the first medicament.

15. The medicament delivery system of claim 14, wherein the control module is adapted to administer the second medicament based on a predetermined second protocol based on the second medicament.

16. The medicament delivery system of claim 11, wherein the cartridge is adapted to be removed after the first medicament and the second medicament are administered.

17. The medicament delivery system of claim 11, wherein the first medicament is different than the second medicament.

18. The medicament delivery system of claim 11, wherein the first medicament is the same as the second medicament.

19. The medicament delivery system of claim 11, wherein the smart user device further includes a linear actuator adapted to engage the primary release mechanism and the secondary release mechanism.

20. A medicament delivery system comprising:
a cartridge including:
a primary autoinjector containing a first medicament;
a primary release mechanism operatively connected to the primary autoinjector;
a secondary autoinjector containing a second medicament; and
a secondary release mechanism operatively connected to the secondary autoinjector;
a smart user device adapted to operatively receive the cartridge, the smart user device including:
a housing (i) coupled to a strap, and (ii) including a receptacle for removably receiving the cartridge therein;
a linear actuator adapted to engage the primary release mechanism and the secondary release mechanism;
a control module (i) operatively connected to the linear actuator, and (ii) adapted to determine the first medicament and the second medicament when the cartridge is inserted into the receptacle;
a first sensor adapted to send data to the control module; and
a second sensor adapted to send data to the control module.
